(19) United States
(12) Patent Application Publication
(10) Pub. No.: US 2015/0087995 A1
(43) Pub. Date: Mar. 26, 2015

(54) BODY INFORMATION OBTAINING DEVICE, BODY INFORMATION OBTAINING METHOD AND BODY INFORMATION OBTAINING PROGRAM

(71) Applicant: CASIO COMPUTER CO., LTD., Tokyo (JP)

(72) Inventor: Kazuaki MURAI, Tokyo (JP)

(73) Assignee: CASIO COMPUTER CO., LTD., Tokyo (JP)

(21) Appl. No.: 14/486,725

(22) Filed: Sep. 15, 2014

(30) Foreign Application Priority Data
Sep. 20, 2013 (JP) 2013-195302

(51) Int. Cl. A61B 5/00; A61B 5/024; A61B 5/11

(52) U.S. Cl. CPC A61B 5/6844; A61B 5/1116; A61B 5/1123; A61B 5/02433
USPC 600/473; 600/595

(57) ABSTRACT

A body information obtaining device, including: a sensor unit which has at least one sensor sensing body information and is wearable on any one of a plurality of wearing positions different from each other of a body of a user; and an actually-wearing position identification unit which identifies an actually-wearing position where the sensor unit is worn among the plurality of wearing positions on basis of a sensing result by the sensor unit.

### CROSS-REFERENCE TO RELATED APPLICATIONS

The entire disclosure of Japanese Patent Application No. 2013-195302 filed on Sep. 20, 2013 including description, claims, drawings, and abstract are incorporated herein by reference in its entirety.

### BACKGROUND OF THE INVENTION

1. Field of the Invention

The present invention relates to a body information obtaining device, a body information obtaining method and a body information obtaining program. Especially, the present invention relates to a body information obtaining device which is wearable on any one of a plurality of types of wearing positions of a body which are different from each other, a body information obtaining method by the body information obtaining device and a body information obtaining program.

2. Description of Related Art

Conventionally, there are pedometers, pulsimeters and bioacoustic sensors as body information obtaining devices which perform sensing regarding movement conditions and biological information of bodies by using various types of sensors and measure the body information of the bodies on the basis of the sensing results.

Such body information obtaining device is described in Japanese Patent Application Laid Open Publication No. 2012-24390, for example.

Here, in a conventional body information obtaining device, a specific wearing position which is one of the positions of the body such as a head, a chest, an arm and a leg is determined in advance as the wearing position to wear the body information obtaining device.

That is, a conventional body information obtaining device is worn around a specific wearing position which is determined in advance for the body information obtaining device, and the body information obtaining device can only perform predetermined sensing on the specific wearing position.

Thus, when sensing is to be performed at various wearing positions of a body, for example, a user needs to prepare a plurality types of body information obtaining devices which are different from each other and correspond to the respective wearing positions.

### SUMMARY OF THE INVENTION

An object of the present invention is to provide a body information obtaining device, a body information obtaining method and a body information obtaining program that a single body information obtaining device can perform automatic sensing corresponding to a wearing position at any one of the plurality of wearing positions of a user around which the body information obtaining device can be worn.

In order to solve the above object, according to one aspect of the present invention, there is provided a body information obtaining device, including: a sensor unit which has at least one sensor sensing body information and is wearable on any one of a plurality of wearing positions different from each other of a body of a user; and a wearing position identification unit which identifies an actually-wearing position where the sensor unit is worn among the plurality of wearing positions on basis of a sensing result by the sensor unit.

According to another aspect of the present invention, there is provided a body information obtaining device, including: a sensor unit which has at least one sensor sensing body information and is wearable on any one of a plurality of wearing positions different from each other of a body of a user; and a sensing control unit which controls the sensor unit to obtain data regarding specific body information that is detectable at a specific wearing position from a specific sensor in the sensor unit which is capable of sensing the specific body information when the sensor unit is identified to be worn on the specific wearing position among the plurality of wearing positions.

According to another aspect of the present invention, there is provided a body information obtaining method by a body information obtaining device for sensing body information, the method including: identifying an actually-wearing position where a sensor is worn among a plurality of wearing positions on basis of a sensing result by a sensor unit which has at least one sensor sensing body information and is wearable on any one of the plurality of wearing positions different from each other of a body of a user.

According to another aspect of the present invention, there is provided a body information obtaining program which makes a computer as a body information obtaining device achieve a function for sensing body information, the function including: identifying an actually-wearing position where a sensor is worn among a plurality of wearing positions on basis of a sensing result by a sensor unit which has at least one type of the sensor sensing the body information and is wearable on the plurality of wearing positions different from each other of a body of a user.

### BRIEF DESCRIPTION OF THE DRAWINGS

The above and other objects, advantages and features of the present invention will become more fully understood from the detailed description given hereinafter and the appended drawings which are given by way of illustration only, and thus are not intended as a definition of the limits of the present invention, and wherein:

FIG. 1A is a perspective view showing an outer appearance of a body information obtaining device;

FIG. 1B is a view showing a state in which body information obtaining devices are worn;

FIG. 2 is a block diagram showing a schematic configuration of the body information obtaining device;

FIG. 3 is a view showing a sensing target storage table;

FIGS. 4A to 4D are views showing acceleration waveform data when the body information obtaining device is worn around an arm;

FIGS. 5A to 5D are views showing acceleration waveform data when the body information obtaining device is worn around a leg;

FIG. 6 is a flow chart showing the flow of body information obtaining processing;

FIGS. 7A to 7D are views showing acceleration vector strengths when the body information obtaining device is worn around an arm;

FIGS. 8A to 8D are views showing acceleration vector strengths when the body information obtaining device is worn around a leg.

### DETAILED DESCRIPTION OF THE PREFERRED EMBODIMENT

Hereinafter, a body information obtaining device, a body information obtaining method and a body information obtaining program according to the present invention will be described in detail with reference to the drawings.

Though the after-mentioned embodiment is provided with various technically preferred limitations to perform the present invention, the scope of the present invention is not limited to the following embodiment and illustrated examples.

### <Outer Configuration>

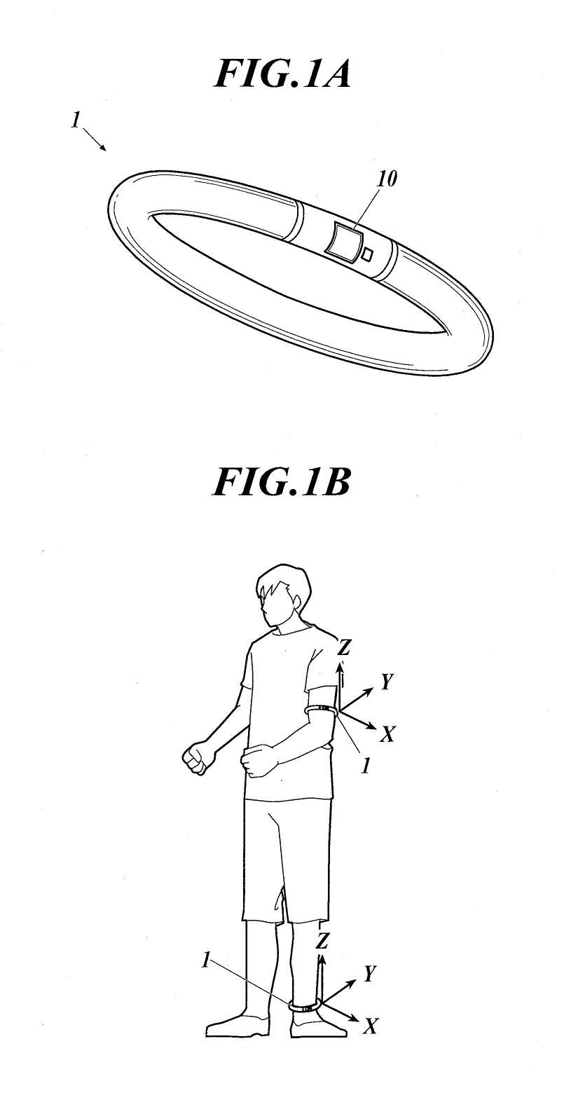

FIG. 1A is an appearance view showing a body information obtaining device 1 in the embodiment.

FIG. 1B is a view showing a state in which a user wears body information obtaining devices 1.

As shown in the drawings, the body information obtaining device 1 is a device for sensing body information including at least any one of movement conditions of a body and biological information such as a pulse rate regarding a body of a user.

As shown in FIG. 1A, the body information obtaining device 1 is configured in the form of a ring having flexibility, for example.

As shown in FIG. 1B, the body information obtaining device 1 is wearable around any one of a plurality of wearing positions (arms and legs in the embodiment) which are different from each other of the body of the user. When the body information obtaining device 1 is formed in a ring shape having flexibility as shown in FIG. 1A, the body information obtaining device 1 can be tightly fitted around any one of arms and ankles as shown in FIG. 1B.

The body information obtaining device 1 includes a button 10.

The button 10 is used when the position where the body information obtaining device 1 is worn is changed, for example, which will be described in detail later.

### <Functional Configuration>

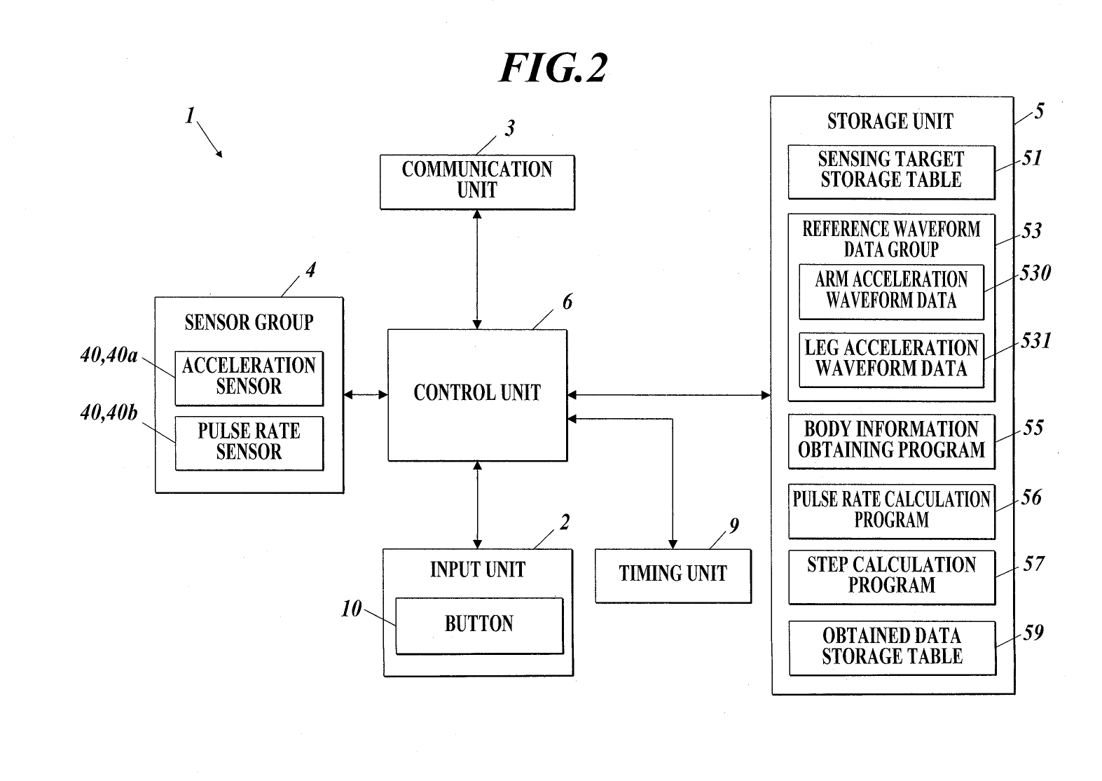

FIG. 2 is a block diagram showing a functional configuration of the body information obtaining device 1 in the embodiment.

As shown in the drawing, the body information obtaining device 1 is configured by including an input unit 2, a communication unit 3, a timing unit 9, a sensor unit 4, a storage unit 5, a control unit 6 and such like.

Among them, the input unit 2 has the above-mentioned button 10 and outputs an operation signal of the button 10 to the control unit 6.

The communication unit 3 is for performing wireless data communication with another body information obtaining device 1 and an external equipment (not shown in the drawings).

The timing unit 9 is for obtaining time information such as elapsed time.

The sensor unit 4 includes at least one type of sensor 40, and in the embodiment, includes an acceleration sensor 40a and a pulse rate sensor 40b.

The acceleration sensor 40a is a sensor for sensing accelerations in three axial directions, for example.

Here, the acceleration sensor 40a can perform comparatively better sensing regarding the movement condition of body for the movement of a leg per step when a user walks or runs in a case in which the user walks or runs while wearing the body information obtaining device 1 around the leg.

On the other hand, in a case in which the user walks or runs while wearing the body information obtaining device 1 around an arm, the acceleration sensor 40a simultaneously performs sensing for the movement condition regarding the arm swing of the user in addition to the sensing for the movement condition regarding the movement of a leg per step of the user.

Thus, sensing result of movement condition regarding the arm swing is noise and the movement condition regarding movement of a leg per step when the user walks or runs cannot be sensed well.

On the other hand, the acceleration sensor 40a can sense the movement condition of an arm well when the body information obtaining device 1 is worn around an arm.

In the embodiment, when the user wears the body information obtaining device 1 correctly, the detecting directions (X direction, Y direction and Z direction) of acceleration by the acceleration sensor 40a are as shown in the above-mentioned FIG. 1B.

The pulse rate sensor 40b is for sensing pulse rates.

The pulse rate sensor 40b in the embodiment can sense a pulse rate well when the body information obtaining device 1 is worn around an arm.

On the other hand, when the body information obtaining device 1 is worn around a leg, the pulse rate sensor 40b cannot sense the pulse rate well since the wearing position is away from the heart.

As such pulse rate sensor 40b, known sensors such as a reflective pulse rate sensor can be used, for example.

The reflective pulse rate sensor is a sensor sensing a pulse rate by using absorption of infrared rays into hemoglobin in blood, and senses the pulse rate by irradiating the blood vessel position with infrared rays, receiving reflected light and detecting the change in strength of reflected light caused by expansion and contraction of the blood vessel.

The storage unit 5 is a memory which stores programs and data for achieving various functions of the body information obtaining device 1 and functions as a working area of the control unit 6.

In the embodiment, in the storage unit 5, a sensing target storage table 51, a reference waveform data group 53, a body information obtaining program 55, a pulse rate calculation program 56, a step calculation program 57, an obtained data storage table 59 and such like are stored.

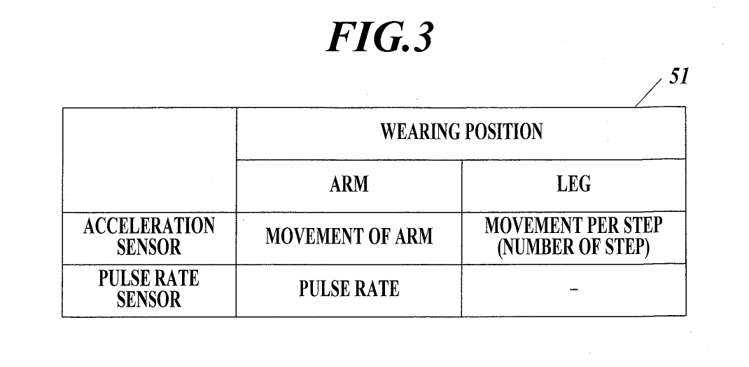

As shown in FIG. 3, in the sensing target storing table 51, the type of sensor 40 used for sensing in the sensor unit 4 and body information which is a target of the sensing performed by the sensor 40 to be used are stored so as to be associated with each other for each of the plurality of wearing positions of the body around which the body information obtaining device 1 can be worn.

The reference waveform data group 53 has arm acceleration waveform data 503 and leg acceleration waveform data 531.

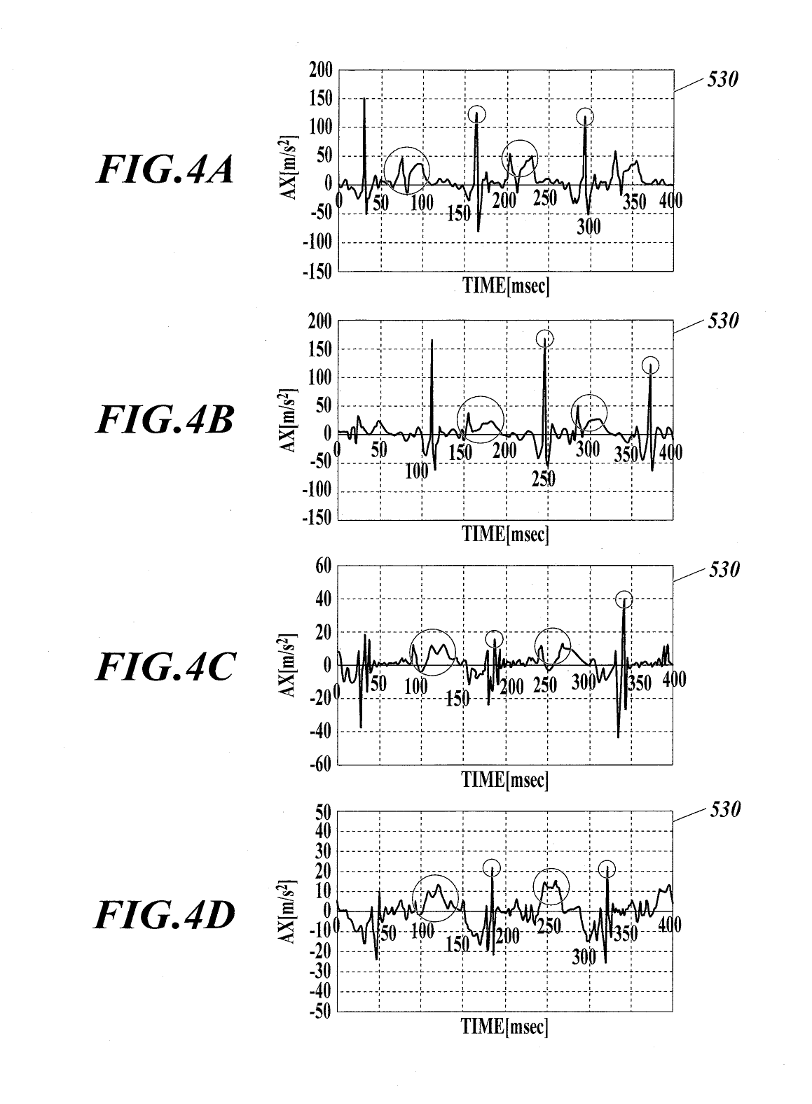

As shown in FIGS. 4A to 4D, the arm acceleration waveform data 530 is data showing waveforms of typical changes to time progress in acceleration values obtained by the acceleration sensor 40a when the body information obtaining device 1 is worn around an arm.

Here, the arm acceleration waveform data 530 shown in FIG. 4A is data showing a waveform of changes to time progress in acceleration in X direction (see FIG. 1B) to be measured by the acceleration sensor 40a when a male athlete runs with the body information obtaining device 1 around his arm.

Similarly, the arm acceleration waveform data 530 shown in FIG. 4B is data showing a waveform of changes to time progress in acceleration to be measured when a female athlete runs with the body information obtaining device 1 around her arm.

The arm acceleration waveform data 530 shown in FIG. 4C is data showing a waveform of changes to time progress in acceleration to be measured when a general male runner runs with the body information obtaining device 1 around his arm.

The arm acceleration waveform data 530 shown in FIG. 4D is data showing a waveform of changes to time progress in acceleration to be measured when a general female runner runs with the body information obtaining device 1 around her arm.

In each piece of the arm acceleration waveform data 530, the portions which are circled in the drawing are characteristic. That is, second largest peaks appear at time bands which are approximately in the middle between a plurality of largest periodic peaks.

The positive and negative of the arm acceleration waveform data 530 may be reversed since the positive and negative of the output signal by the acceleration sensor 40a is reversed according to the direction in which the body information device 1 is worn.

It is preferable that any piece of the arm acceleration waveform data 530 shown in FIGS. 4A to 4D is selected when the user performs initial setting.

As such arm acceleration waveform data 530, data obtained by a user himself/herself may also be used.

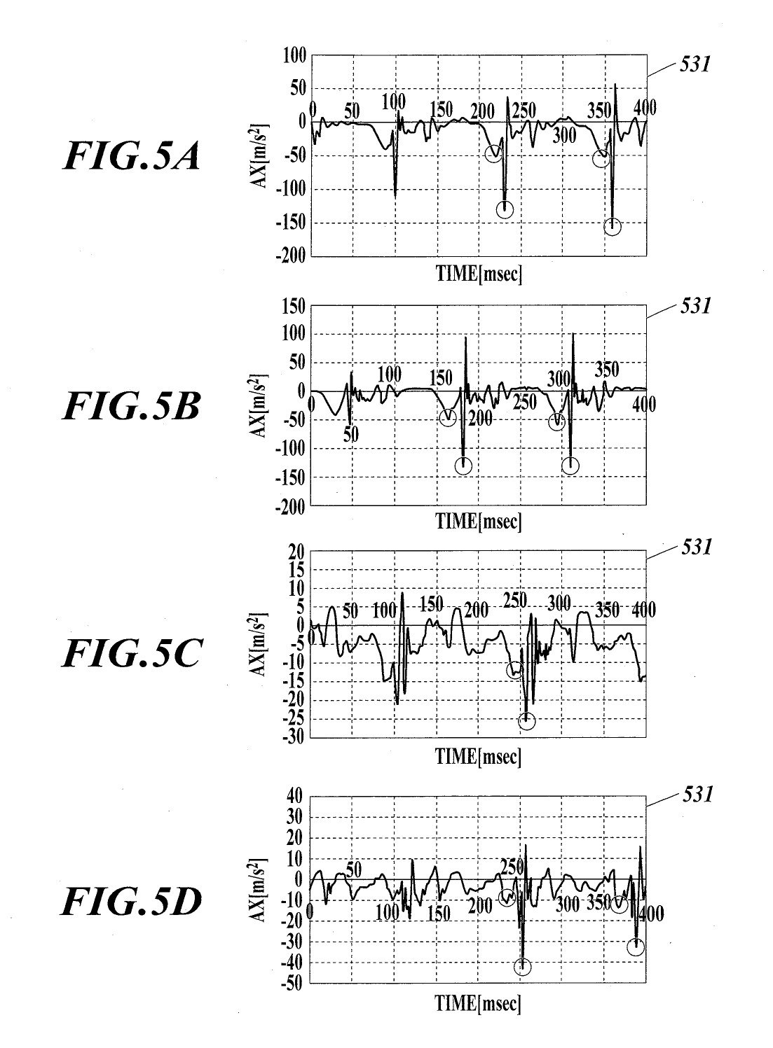

As shown in FIGS. 5A to 5D, the leg acceleration waveform data 531 is data showing waveforms of typical changes to time progress in acceleration values to be obtained by the acceleration sensor 40a when the body information obtaining device 1 is worn around a leg.

Here, the leg acceleration waveform data 531 shown in FIG. 5A is data showing a waveform of changes to time progress in acceleration in X direction (see FIG. 1B) to be measured by the acceleration sensor 40a when a male athlete runs with the body information obtaining device 1 around his leg.

Similarly, the leg acceleration waveform data 531 shown in FIG. 5B is data showing a waveform of changes to time progress in acceleration to be measured when a female athlete runs with the body information obtaining device 1 around her leg.

The leg acceleration waveform data 531 shown in FIG. 5C is data showing a waveform of changes to time progress in acceleration to be measured when a general male runner runs with the body information obtaining device 1 around his leg.

The leg acceleration waveform data 531 shown in FIG. 5D is data showing a waveform of changes to time progress in acceleration to be measured when a general female runner runs with the body information obtaining device 1 around her leg.

In each piece of the leg acceleration waveform data 531, the portions which are circled in the drawing are characteristic. That is, second largest peaks appear immediately before largest peaks, respectively.

The positive and negative of the leg acceleration waveform data 531 may be reversed since the output signal by the acceleration sensor 40a is reversed in positive and negative according to the direction in which the body information obtaining device 1 is worn.

It is preferable that any piece of leg acceleration waveform data 531 shown in FIGS. 5A to 5D is selected when the user performs initial setting.

As such leg acceleration waveform data 531, data obtained by a user himself/herself may also be used.

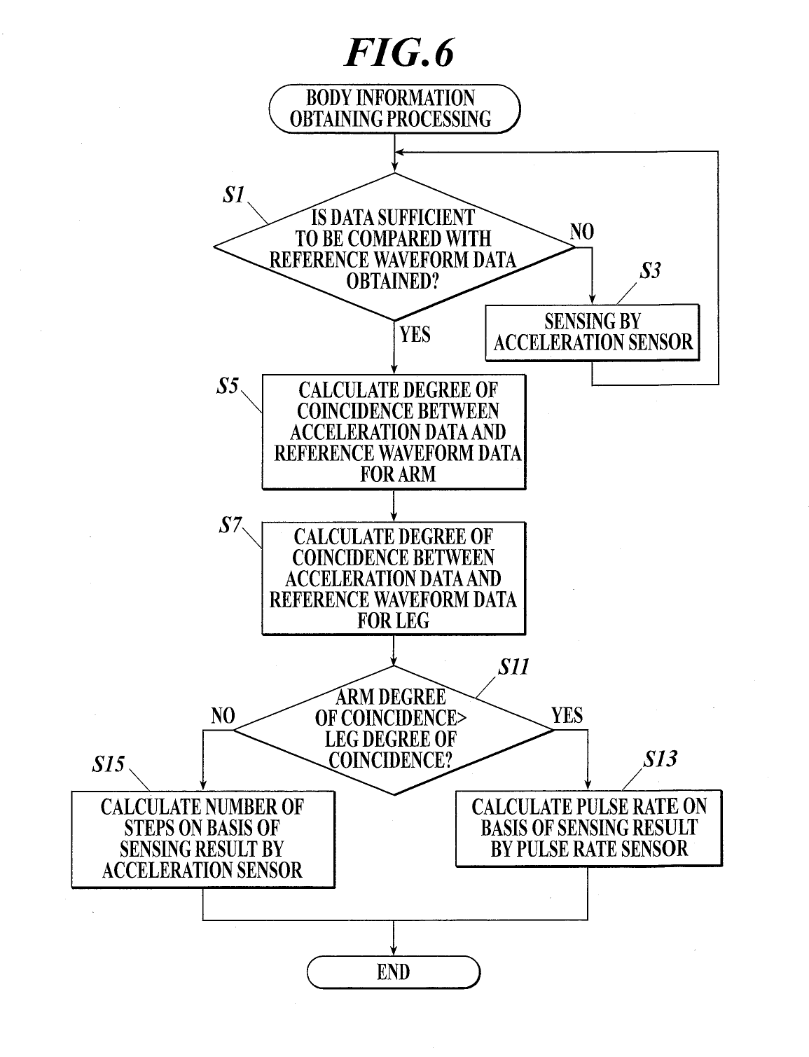

The body information obtaining program 55 is for executing after-mentioned body information obtaining processing (see FIG. 6) by the control unit 6.

The pulse rate calculation program 56 is a program for calculating a pulse rate from the sensing result by the pulse rate sensor 40b when the body information obtaining device 1 is worn around an arm. Known programs can be used as such pulse rate calculation program 56.

The step calculation program 57 is a program for calculating the number of steps from the sensing result by the acceleration sensor 40a when the body information obtaining device 1 is worn around a leg. Known programs can be used as such step calculation program 57.

In the obtained data storage table 59, the data (raw data) of sensing result by the sensor unit 4 is stored to be accumulated so as to be associated with the type of sensor 40 which performed the sensing, a tag indicating the wearing position (hereinafter, called actually-wearing position) around which the body information obtaining device 1 was worn during the sensing, and time when the sensing was performed.

In the obtained data storage table 59 in the embodiment, data regarding the number of steps and the pulse rate calculated from the data of sensing result is stored.

The control unit 6 centrally controls the units of the body information obtaining device 1.

Specifically, the control unit 6 opens a program specified among various programs stored in the storage unit 5 and executes various types of processing in cooperation with the opened program.

The control unit 6 stores the processing result in the storage unit 5 and appropriately outputs the processing result to the communication unit 3.

### [Operation]

Next, body information obtaining processing executed by the body information obtaining device 1 will be described with reference to the drawings.

FIG. 6 is a flow chart for explaining operations of the body information obtaining processing.

After the body information obtaining device 1 is activated, when the button 10 is operated, the body information obtaining program 55 is read out from the storage unit 5 and opened appropriately, and as a result, the body information obtaining processing is executed in cooperation between the body information obtaining program 55 and the control unit 6.

As shown in the drawing, of the body information obtaining processing, the control unit 6 first determines whether data is obtained for a sufficient amount to be compared with the arm acceleration waveform data 530 and the leg acceleration waveform data 531 in the reference waveform data group 53 (step S1).

Here, the determination regarding whether the amount of obtained data is sufficient is performed by, for example, comparing the time width of obtained data with time width of arm acceleration waveform data 530 and the leg acceleration waveform data 531 in the reference waveform data group 53 (400 msec in the arm acceleration waveform data 530 shown in FIGS. 4A to 4D and the leg acceleration waveform data 531 shown in FIGS. 5A to 5D).

That is, if the time width of obtained data is the same as or longer than the time width of arm acceleration waveform data 530 and the leg acceleration waveform data 531, the obtained data is determined to be sufficient. If the time width of obtained data is shorter than the time width of arm acceleration waveform data 530 and the leg acceleration waveform data 531, the obtained data is determined to be not sufficient.

If it is not determined that the sufficient amount of data is obtained in step S1 (step S1; NO), the control unit 6 continuously performs sensing by the acceleration sensor 40a and stores the data (raw data) which is the sensing result in the obtained data storage table 59 so as to be associated with the current time (step S3) and shifts to step S1. Thus, the sensing by the acceleration sensor 40a is continued until sufficient amount of data is obtained.

On the other hand, if it is determined that sufficient amount of data is obtained in step S1 (step S1; YES), the control unit 6 performs pattern matching between the data of sensing result obtained by the acceleration sensor 40a (in the embodiment, acceleration waveform data in X direction (see FIG. 1B)) and each piece of the arm acceleration waveform data 530 (and the positive-negative reversed data of the arm acceleration waveform data 530) to calculate the degree of matching (correlation coefficient) (step S5).

Next, the control unit 6 performs pattern matching between the data of sensing result obtained by the calculation sensor 40a (in the embodiment, acceleration waveform data in X direction (see FIG. 1B)) and each piece of the leg acceleration waveform data 531 (and the positive-negative reversed data of the leg acceleration waveform data 531) to calculate the degree of matching (correlation coefficient) (step S7).

Next, the control unit 6 compares the degree of matching with respect to the arm acceleration waveform data 530 (and the positive-negative reversed data of arm acceleration waveform data 530) with the degree of matching with respect to the leg acceleration waveform data 531 (and the positive-negative reversed data of leg acceleration waveform data 531), and determines whether the former degree of matching is higher than the latter degree of matching (step S11).

By comparing the acceleration waveform of sensing result by the acceleration sensor 40a with each of the acceleration waveforms which could be obtained by the acceleration sensor 40a worn around an arm and a leg in such way, the control unit 6 can identify, among the plurality of wearing positions of a body, the actually-wearing position where the body information obtaining device 1 is worn.

If it is determined that the degree of matching (correlation coefficient) with respect to the arm acceleration waveform data 530 is the higher in step S11 (step S11; YES), the control unit 6 identifies that the body information obtaining device 1 is worn around an arm.

In such case, data (raw data) regarding pulse rate is obtained from the pulse rate sensor 40b on the basis of the type of sensor 40 used for the sensing when worn around an arm and body information regarding the sensing target to be sensed by the sensor 40 which are stored in the sensing target storage table 51.

Then, the control unit 6 calculates the pulse rate from the data of sensing result by the pulse rate sensor 40b by using the pulse rate calculation program 56, stores the raw data and the pulse rate data in the obtained data storage table 59 (step S13), and ends the body information obtaining processing.

In this step S13, the control unit 6 stores the raw data (data of sensing result by the pulse rate sensor 40b) stored in the obtained data storage table 59 so as to be associated with a tag indicating “arm” as the actually-wearing position and the time when the sensing was performed.

Furthermore, in the step S13, the control unit 6 obtains data (raw data) regarding arm movement from the acceleration sensor 40a and stores the raw data, the tag indicating “arm” as the actually-wearing position and the time when the sensing was performed in the obtained data storage table 59 so as to be associated with each other.

In this step S13, in a case in which the body information obtaining device 1 can communicate with second body information obtaining device 1 via the communication unit 3, the control unit 6 may identify that the second body information obtaining device 1 is worn around an arm similarly to the first body information obtaining device 1 and make the second body information obtaining device 1 perform the same processing as that of the first body information obtaining device 1.

On the other hand, if it is not determined that the degree of matching (correlation coefficient) with respect to the arm acceleration waveform data 530 is the higher in step S11 (step S11; NO), the control unit 6 identifies that the body information obtaining device 1 is worn around a leg.

In such case, data (raw data) regarding movement of walking or running per step is obtained from the acceleration sensor 40a on the basis of the type of sensor 40 used for sensing when worn around a leg and body information regarding the sensing target to be sensed by the sensor 40 which are stored in the sensing target storage table 51.

Then, the control unit 6 calculates the number of steps from the data of sensing result by the acceleration sensor 40a by using the step calculation program 57 and stores the raw data and the step data in the obtained data storage table 59 (step S15), and ends the body information obtaining processing.

In this step S15, the control unit 6 stores the raw data (data of sensing result by the acceleration sensor 40a) stored in the obtained data storage table 59 so as to be associated with a tag indicating “leg” as the actually-wearing position and the time when the sensing was performed.

In this step S15, the control unit 6 controls the pulse rate sensor 40b not to perform sensing.

In this step S15, in a case in which the body information obtaining device 1 can communicate with second body information obtaining device 1 via the communication unit 3, the control unit 6 may identify that the second body information obtaining device 1 is worn around a leg similarly to the first body information obtaining device 1 and make the second body information obtaining device 1 perform the same processing as that of the first body information obtaining device 1.

As described above, according to the body information obtaining device 1 in the embodiment, as shown in FIG. 6, on the basis of sensing result by any one of the sensors 40 in the sensor unit 4, the actually-wearing position where the body information obtaining device 1 is worn is identified among a plurality of wearing positions of a body around which the body information obtaining device 1 can be worn. Then, data regarding body information of the sensing target associated with the actually-wearing position is obtained from the sensor 40 which is the type of sensor associated with the actually-wearing position.

Thus, for example, in a case in which sensing is to be performed on a plurality of wearing positions of a body to obtain body information which can be sensed at the respective wearing positions, it is possible to automatically perform sensing of body information corresponding to the respective wearing positions with the body information obtaining device 1 worn around the respective wearing positions by wearing the same body information obtaining device 1 around the respective wearing positions of the body without preparing a plurality of body information obtaining devices which are different from each other and corresponding to the respective wearing positions nor operating to set body information of sensing targets with respect to the body information obtaining devices worn around the respective wearing positions.

The actually-wearing position among the wearing positions is identified by comparing the acceleration waveform of sensing result by the acceleration sensor 40a with typical acceleration waveforms which could be obtained by the acceleration sensor 40a at a plurality of wearing positions, and thus, the actually-wearing position can be accurately identified.

Since the sensing result by the sensor 40 and the tag indicating the actually-wearing position are stored in the obtained data storage table 59 so as to be associated with each other, data regarding body information corresponding to the actually-wearing position can be obtained from the raw data of sensing result.

It goes without saying that changes can be appropriately made within the scope of the present invention with respect to the detailed configuration and detailed operation of the components of the body information obtaining device 1 in the embodiment.

For example, the body information obtaining device according to the present invention may be applied to an electronic device such as a mobile phone, a PDA (Personal Digital Assistant) and a game machine as long as it is formed to be wearable around a plurality of wearing positions of a body and performs sensing of body information regarding the body. In such case, the body information obtaining device 1 may be formed to be a thin plate and tightly fitted on a body by a separate stretching band.

In the embodiment, among the sensing results by the acceleration sensor 40a, the acceleration waveform in X direction is compared with typical acceleration waveforms (arm acceleration waveform data 530 and leg acceleration waveform data 531) which could be obtained by the acceleration sensor 40a around an arm and a leg, and thereby the actually-wearing position among the plurality of wearing positions is identified.

In addition to (or instead of) this method, the actually-wearing position among the plurality of wearing positions may be identified on the basis of peak values of acceleration vector strengths (square root of a total value of squares of accelerations in X, Y and Z directions) obtained from the sensing result by the acceleration sensor 40a.

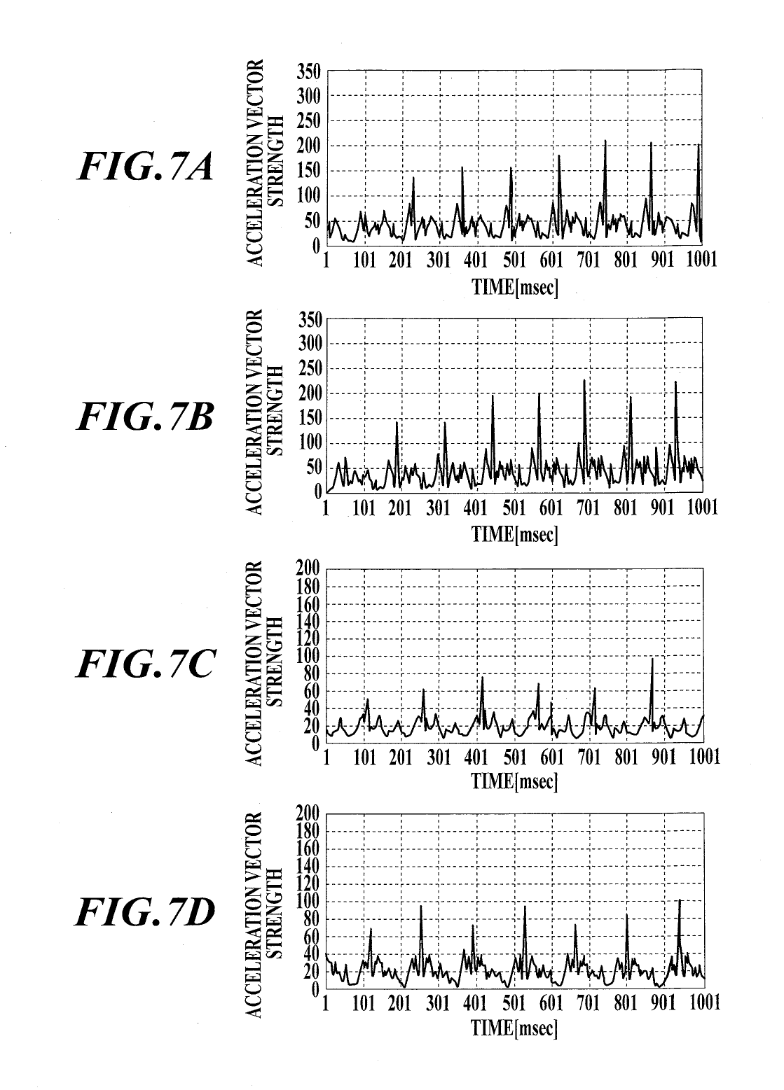

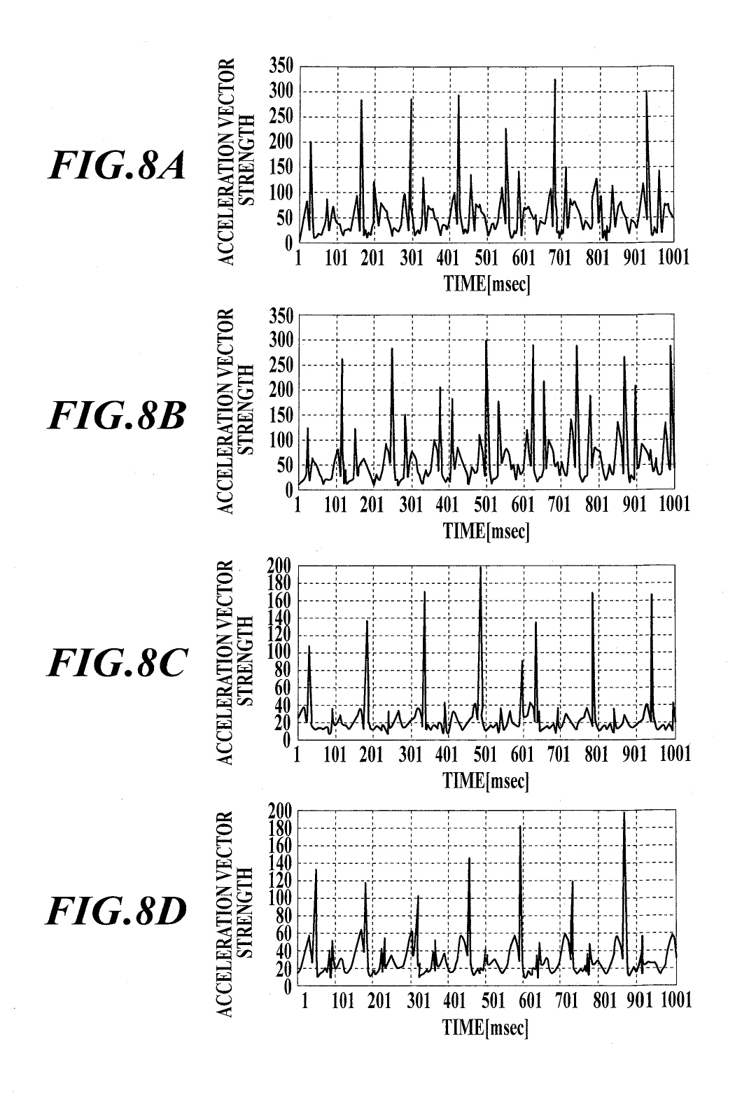

That is, FIGS. 7A to 7D are views showing acceleration vector strengths when the body information obtaining device 1 is worn around an arm, and FIGS. 8A to 8D are views showing acceleration vector strengths when the body information obtaining device 1 is worn around a leg.

Here, the acceleration vector strengths shown in FIGS. 7A and 8A are acceleration vector strengths to be measured by the acceleration sensor 40a when the male athlete runs with the body information obtaining device 1 around his arm and leg, respectively.

Similarly, the acceleration vector strengths shown in FIGS. 7B and 8B are acceleration vector strengths to be measured when the female athlete runs with the body information obtaining device 1 around her arm and leg, respectively.

The acceleration vector strengths shown in FIGS. 7C and 8C are acceleration vector strengths to be measured when the general male runner runs with the body information obtaining device 1 around his arm and leg, respectively.

The acceleration vector strengths shown in FIGS. 7D and 8D are acceleration vector strengths to be measured when the general female runner runs with the body information obtaining device 1 around her arm and leg, respectively.

As shown in FIGS. 7A to 7D and 8A to 8D, the acceleration vector strengths when the body information obtaining device 1 is worn around an arm have smaller peak values than those of the acceleration vector strengths when the body information obtaining device 1 is worn around a leg.

Thus, when peak values of these acceleration vector strengths are obtained from the user in advance, the control unit 6 can identify whether the actually-wearing position is an arm or leg by comparing the peak values with peak values newly obtained from the acceleration sensor 40a.

The actually-wearing position is identified among the plurality of wearing positions on the basis of the sensing result by the acceleration sensor 40a in the embodiment; however, the actually-wearing position may be identified on the basis of the sensing result by the pulse rate sensor 40b.

That is, the pulse rate sensor 40b can perform sensing of pulse rate well when the body information obtaining device 1 is worn around an arm. However, the pulse rate sensor 40b cannot perform sensing of pulse rate well when the body information obtaining device 1 is worn around a leg. Thus, the control unit 6 identifies whether the actually-wearing position is an arm or leg by detecting whether the pulse rate was obtained on the basis of sensing result by the pulse rate sensor 40b. Also in this case, whether the actually-wearing position is an arm or leg can be accurately identified.

Though the sensor unit 4 includes the acceleration sensor 40a and the pulse rate sensor 40b in the embodiment, the sensor unit 4 may include only the acceleration sensor 40a.

Alternatively, the sensor unit 4 may include an ultrasonic sensor in addition to (or instead of either one of) the acceleration sensor 40a and the pulse rate sensor 40b.

When the sensor unit 4 includes the ultrasonic sensor, the control unit 6 can identify the actually-wearing position among the plurality of wearing positions by detecting the distance from the ground to the body information obtaining device 1 on the basis of the sensing result by the ultrasonic sensor. Even in this case, the actually-wearing position among the plurality of wearing positions can be accurately identified.

Though the body information obtaining device 1 can be worn around an arm and a leg in the above embodiment, the body information obtaining device 1 may be further wearable around other wearing positions of the body such as a chest and a neck.

Though several embodiments of the present invention have been described above, the scope of the present invention is not limited to the above embodiments, and includes the scope of inventions, which is described in the scope of claims, and the scope equivalent thereof.

What is claimed is:

1. A body information obtaining device, comprising:
a sensor unit which has at least one sensor sensing body information and is wearable on any one of a plurality of wearing positions different from each other of a body of a user; and
a wearing position identification unit which identifies an actually-wearing position where the sensor unit is worn among the plurality of wearing positions on basis of a sensing result by the sensor unit.

2. The body information obtaining device according to claim 1, wherein
the sensor unit has an acceleration sensor which senses acceleration; and
the wearing position identification unit identifies the actually-wearing position among the plurality of wearing positions on basis of comparison between an observed waveform showing changes to time progress in the acceleration sensed by the acceleration sensor and each reference waveform of a plurality of reference waveforms showing typical changes to time progress in accelerations to be obtained by sensing at the respective plurality of wearing positions with the acceleration sensor.

3. The body information obtaining device according to claim 2, further comprising a reference waveform data storage unit in which the reference waveforms are stored,
wherein
the wearing position identification unit calculates a correlation coefficient between the observed waveform and each of the reference waveforms by performing pattern matching between the observed waveform and each of the plurality of reference waveforms stored in the reference waveform data storage unit and identifies that a wearing position which has a largest correlation coefficient among the plurality of wearing positions is the actually-wearing position.

4. The body information obtaining device according to claim 1, wherein
the sensor unit has a pulse rate sensor which senses a pulse rate; and
the wearing position identification unit identifies the actually-wearing position among the plurality of wearing positions on basis of a sensing result of the pulse rate by the pulse rate sensor.

5. The body information obtaining device according to claim 1, wherein
the sensor unit has an ultrasonic sensor which detects a distance from ground to the sensor unit; and
the wearing position identification unit identifies the actually-wearing position among the plurality of wearing positions on basis of a value of the distance detected by the ultrasonic sensor.

6. The body information obtaining device according to claim 1, further comprising:
a sensing target storage unit in which a type of a sensor in the sensor unit used for sensing body information and a type of the body information which is a target of the sensing are stored so as to be associated with each other for each of the wearing positions; and
a sensing control unit which controls the sensor unit by setting a sensor used for sensing in the sensor unit and body information to be sensed by the sensor on basis of identification by the wearing position identification unit determining the actually-wearing position among the plurality of wearing positions and information stored in the sensing target storage unit.

7. The body information obtaining device according to claim 6, wherein, when the wearing position identification unit identifies that the actually-wearing position is a specific wearing position among the plurality of wearing positions and sensing of specific body information at the specific wearing position using a specific sensor in the sensor unit is stored in the sensing target storage unit, the sensing control unit controls the sensor unit to use the specific sensor in the sensor unit to sense the specific body information and obtain data.

8. The body information obtaining device according to claim 6, wherein
the sensor unit has an acceleration sensor which senses acceleration,
the plurality of wearing positions include an arm and a leg of the user,
when the wearing position identification unit identifies that the actually-wearing position is the arm, the sensing control unit controls the sensor unit to use the acceleration sensor and sense body information regarding movement of the arm to obtain data from the acceleration sensor, and
when the wearing position identification unit identifies that the actually-wearing position is the leg, the sensing control unit controls the sensor unit to use the acceleration sensor and sense body information regarding movement per step of walking or running to obtain data from the acceleration sensor.

9. The body information obtaining device according to claim 6, wherein
the sensor unit has a pulse rate sensor which senses a pulse rate,
the plurality of wearing positions include an arm and a leg of the user,
when the wearing position identification unit identifies that the actually-wearing position is the arm, the sensing control unit controls the sensor unit to use the pulse rate sensor and sense the pulse rate at the arm to obtain data, and
when the wearing position identification unit identifies that the actually-wearing position is the leg, the sensing control unit controls the sensor unit not to perform sensing of the pulse rate using the pulse rate sensor.

10. A body information obtaining device, comprising:
a sensor unit which has at least one sensor sensing body information and is wearable on any one of a plurality of wearing positions different from each other of a body of a user; and
a sensing control unit which controls the sensor unit to obtain data regarding specific body information that is detectable at a specific wearing position from a specific sensor in the sensor unit which is capable of sensing the specific body information when the sensor unit is identified to be worn on the specific wearing position among the plurality of wearing positions.

11. The body information obtaining device according to claim 10, further comprising a wearing position identification unit which identifies an actually-wearing position where the sensor unit is worn among the plurality of wearing positions on basis of a sensing result by the sensor unit.

12. The body information obtaining device according to claim 11, wherein
the sensor unit has an acceleration sensor which senses acceleration; and
the wearing position identification unit identifies the actually-wearing position among the plurality of wearing positions on basis of comparison between an observed waveform showing changes to time progress in the acceleration sensed by the acceleration sensor and each reference waveform of a plurality of reference waveforms showing typical changes to time progress in accelerations to be obtained by sensing at the respective plurality of wearing positions with the acceleration sensor.

13. The body information obtaining device according to claim 11, wherein
the sensor unit has a pulse rate sensor which senses a pulse rate; and
the wearing position identification unit identifies the actually-wearing position among the plurality of wearing positions on basis of a sensing result of the pulse rate by the pulse rate sensor.

14. The body information obtaining device according to claim 11, wherein
the sensor unit has an ultrasonic sensor which detects a distance from ground to the sensor unit; and
the wearing position identification unit identifies the actually-wearing position among the plurality of wearing positions on basis of a value of the distance detected by the ultrasonic sensor.

15. A body information obtaining method by a body information obtaining device for sensing body information, the method comprising:
identifying an actually-wearing position where a sensor is worn among a plurality of wearing positions on basis of a sensing result by a sensor unit which has at least one sensor sensing body information and is wearable on any one of the plurality of wearing positions different from each other of a body of a user.

16. The body information obtaining method according to claim 15, wherein
the sensor unit has an acceleration sensor which senses acceleration; and
in the identifying of the actually-wearing position, the actually-wearing position among the plurality of wearing positions is identified on basis of comparison between an observed waveform showing changes to time progress in the acceleration sensed by the acceleration sensor and each reference waveform of a plurality of reference waveforms showing typical changes to time progress in accelerations to be obtained by sensing at the respective plurality of wearing positions with the acceleration sensor.

17. The body information obtaining method according to claim 15, wherein
the sensor unit has a pulse rate sensor which senses a pulse rate; and
in the identifying of the actually-wearing position, the actually-wearing position among the plurality of wearing positions is identified on basis of a sensing result of the pulse rate by the pulse rate sensor.

18. The body information obtaining method according to claim 15, wherein
the sensor unit has an ultrasonic sensor which detects a distance from ground to the sensor unit; and
in the identifying of the actually-wearing position, the actually-wearing position among the plurality of wearing positions is identified on basis of a value of the distance detected by the ultrasonic sensor.

19. The body information obtaining method according to claim 15, further comprising controlling the sensor unit by setting a sensor used for sensing in the sensor unit and body information to be sensed by the sensor on basis of identification identifying the actually-wearing position among the plurality of wearing positions and information stored in a sensing target storage unit in which a type of a sensor in the sensor unit used for sensing body information and a type of the body information which is a target of the sensing are stored so as to be associated with each other for each of the wearing positions.

20. A body information obtaining program which makes a computer as a body information obtaining device achieve a function for sensing body information, the function comprising:
identifying an actually-wearing position where a sensor is worn among a plurality of wearing positions on basis of a sensing result by a sensor unit which has at least one type of the sensor sensing the body information and is wearable on the plurality of wearing positions different from each other of a body of a user.
